(19) United States
(12) Patent Application Publication
(10) Pub. No.: US 2013/0345550 A1
(43) Pub. Date: Dec. 26, 2013

(54) SYSTEMS AND METHODS FOR LOCALIZING AN OPAQUE MEDICAL DEVICE WITH NUCLEAR MEDICINE IMAGING

(71) Applicants: Tzachi Rafaeli, Shlmsnit (IL); Ira Micah Blevis, Zicron Yaakov (IL); Yaron Hefetz, Kibbutz Alonim (IL)

(72) Inventors: Tzachi Rafaeli, Shlmsnit (IL); Ira Micah Blevis, Zicron Yaakov (IL); Yaron Hefetz, Kibbutz Alonim (IL)

(21) Appl. No.: 13/532,642

(22) Filed: Jun. 25, 2012

(51) Int. Cl. A61B 6/02; A61B 10/02

(52) U.S. Cl. 600/424

(57) ABSTRACT

Systems and methods for localizing a medical device with nuclear medicine imaging are provided. One system includes a medical tool having a body with a length and configured to be inserted within an object. The medical tool also includes one or more radiation opaque regions along at least a portion of the length of the body, wherein the radiation opaque regions block gamma ray emission from within the object.

### BACKGROUND OF THE INVENTION

The subject matter disclosed herein relates generally to nuclear medicine (NM) imaging systems, and more particularly to localizing an opaque medical device, such as a biopsy tool, with the NM imaging systems.

Nuclear Medicine (NM) imaging systems, such as Positron Emission Tomography (PET) and Single Photon Emission Computed Tomography (SPECT) systems may be used to perform biopsies or other procedures. For example, biopsy procedures may be performed for breast cancer detection. However, these procedures are invasive and not pleasant. Accordingly, if the biopsy needle is not positioned accurately within lesion (e.g., in order to take a sample), the needle may need to be manipulated within the breast or reinserted, adding time and discomfort to the patient.

Known biopsy procedures for PET and SPECT use dedicated devices having a radioactive rod inserted into the biopsy tool so that the tool is visible in images acquired by the PET and SPECT systems. The radioactive rod typically contains a radioactive material with a long life. The biopsy tool with the radioactive rod is a biohazard once used and until the tool is sterilized. Thus, protection from the radioactivity must be used when cleaning the tool. The biopsy tool also requires proper storage to prevent exposure to radiation. Additionally, the biopsy tool also must be sterilized after each use, which becomes more difficult because of the potential exposure to the radiation emitted by the radioactive rod. Thus, the handling of known biopsy tools for PET and SPECT imaging require special handling and storage.

### BRIEF DESCRIPTION OF THE INVENTION

In various embodiments, a medical tool for use in nuclear medicine imaging is provided. The medical tool includes a body having a length and configured to be inserted within an object. The medical tool also includes one or more radiation opaque regions along at least a portion of the length of the body, wherein the radiation opaque regions block gamma ray emission from within the object.

In other various embodiments, a nuclear medicine (NM) imaging system is provided that includes a gantry, a first nuclear medicine detector mounted to the gantry and a second nuclear medicine detector mounted to the gantry, wherein the first and second nuclear medicine detectors are configured to acquire planar NM images. The NM imaging system also includes a biopsy guiding tool and a biopsy needle configured to be guided within an object between the first and second nuclear medicine detectors, wherein the biopsy needle has one or more radiation opaque properties.

In still other various embodiments, a method for localizing a medical tool in nuclear medicine (NM) imaging is provided. The method includes acquiring NM data of a region of interest within an object, wherein the region of interest includes a medical tool having one or more radiation opaque regions. The method also includes identifying one or more areas of a concentration of radioactivity in an image formed from the NM data, wherein the one or more areas have a lower concentration than at least one of a lesion radioactivity concentration and a background radioactivity concentration. The one or more areas correspond to the one or more radiation opaque regions. The method further includes localizing the medical tool using the identified one or more areas having the lower concentration of radioactivity.

### BRIEF DESCRIPTION OF THE DRAWINGS

FIG. 1 is a schematic block diagram of an exemplary nuclear medicine (NM) imaging system constructed in accordance with various embodiments.

FIG. 2 is a diagram of a detector configuration for acquiring planar images in accordance with various embodiments.

FIG. 3 is a diagram illustrating an image acquired in accordance with various embodiments for localizing a medical tool.

FIG. 4 are images acquired in accordance with various embodiments for localizing a medical tool.

FIG. 5 is a diagram illustrating identification of a medical tool in accordance with one embodiment.

FIG. 6 is a diagram of a medical tool formed in accordance with an embodiment.

FIG. 7 is a diagram illustrating identification of a medical tool in accordance with another embodiment.

FIG. 8 is a diagram illustrating scattered photons used in accordance with one embodiment.

FIG. 9 is a graph illustrating scatter photon energy.

FIG. 10 is a flowchart of a method for localizing a medical tool in accordance with various embodiments.

### DETAILED DESCRIPTION OF THE INVENTION

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Also as used herein, the phrase “reconstructing an image” is not intended to exclude embodiments in which data representing an image is generated, but a viewable image is not. Therefore, as used herein the term “image” broadly refers to both viewable images and data representing a viewable image. However, many embodiments generate, or are configured to generate, at least one viewable image.

Various embodiments described herein provide systems and methods for localizing a medical device, for example, a biopsy tool in Nuclear Medicine (NM) imaging. For example, biopsy needle localization may be provided in accordance with various embodiments during a biopsy procedure using Positron Emission Tomography (PET) and/or Single Photon Emission Computed Tomography (SPECT) imaging. The medical device of various embodiments may be localized without use of a radioactive source within the device. At least one technical effect of various embodiments is a reduced exposure of radiation to the patient and/or operator. Also, by practicing various embodiments, a less expensive, optionally disposable marker may be used, such that there is no re-sterilization or radioactive shielding.

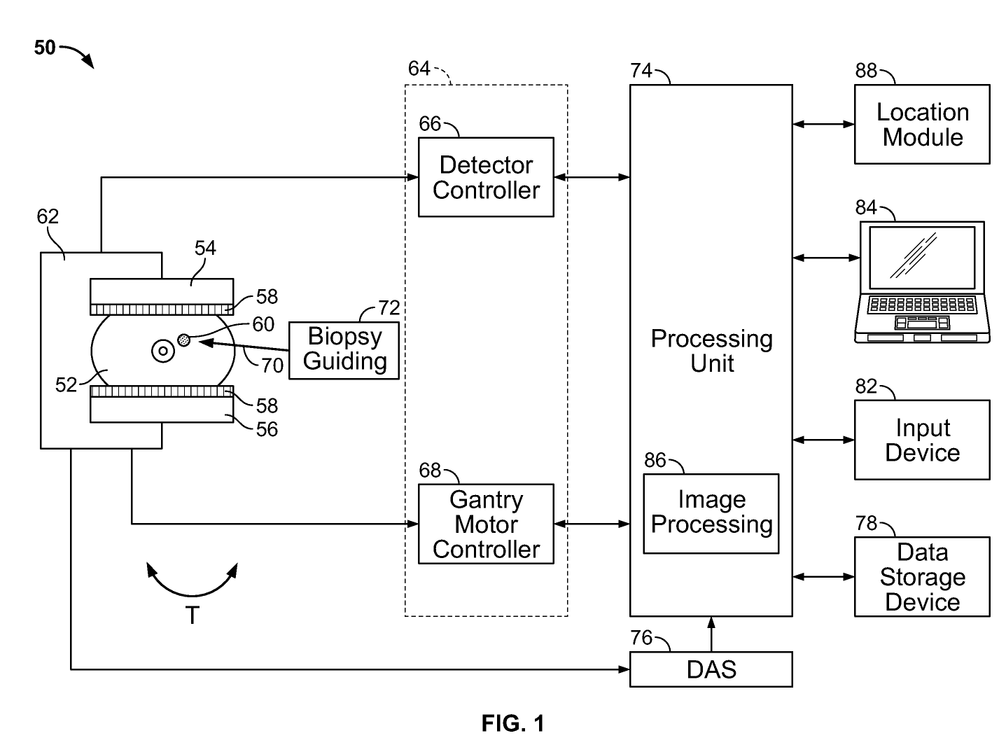

FIG. 1 is a schematic block diagram of one example of an NM imaging system 50 in which various embodiments may be implemented. The NM imaging system 50 includes first and second detectors 54 and 56 mounted on a gantry 62 (or other support structure) that allow movement and positioning of the first and second detectors 54 and 56. The first and second detectors 54 and 56 are configured in some embodiments as a pair of imaging detectors 54 and 56 that are each independently and individually controllable, including movement of the detectors 54 and 56 (e.g., rotation about one or more axis). For example, the first and/or second detectors 54 and 56 may be titled along an axis transverse to a breast 52 (as illustrated by the arrow T) or may be tilted along an axis generally perpendicular to the body of an imaged patient as described in more detail herein. It should be noted that the detectors 54 and 56 may be titled in the same or different directions and at the same or different angles.

The first and second detectors 54 and 56 are arranged and operate to provide two two-dimensional (2D) images of the breast 52. The first and second detectors 54 and 56 are illustrated as planar single photon imaging detectors, however, other configurations may be provided. In various embodiments, the first and second detectors 54 and 56 may be formed of cadmium zinc telluride (CZT) tiles or may any type of 2D pixilated detector, or a scintillator based detector. In various embodiments, the detectors 54 and 56 also include collimators 58 coupled thereto on a detection surface of the detectors 54 and 56, which are illustrated as parallel hole collimators 58. However, other types of collimators may be provided, such as diverging, converging, pinhole, cone-beam, fan-beam or slanted collimators, among others.

Each detector 54 and 56 captures a 2D image that may be defined by the x and y location of a pixel and a detector number. At least one of the detectors 54 and 56 may change orientation relative to a stationary or movable gantry 62. Because the detectors 54 and 56 are registered, features appearing at a given location in one detector 54 and/or 56 can be correctly located and the data correlated in the other detector 54 and/or 56, for example, as described in U.S. Patent Application Publication 2011/0268339, entitled “System and Method for Determining A Location of a Lesion In A Breast” and/or U.S. Patent Application Publication 2010/0261997, entitled “System and Method for Molecular Breast Imaging with Biopsy Capability and Improved Tissue Coverage”.

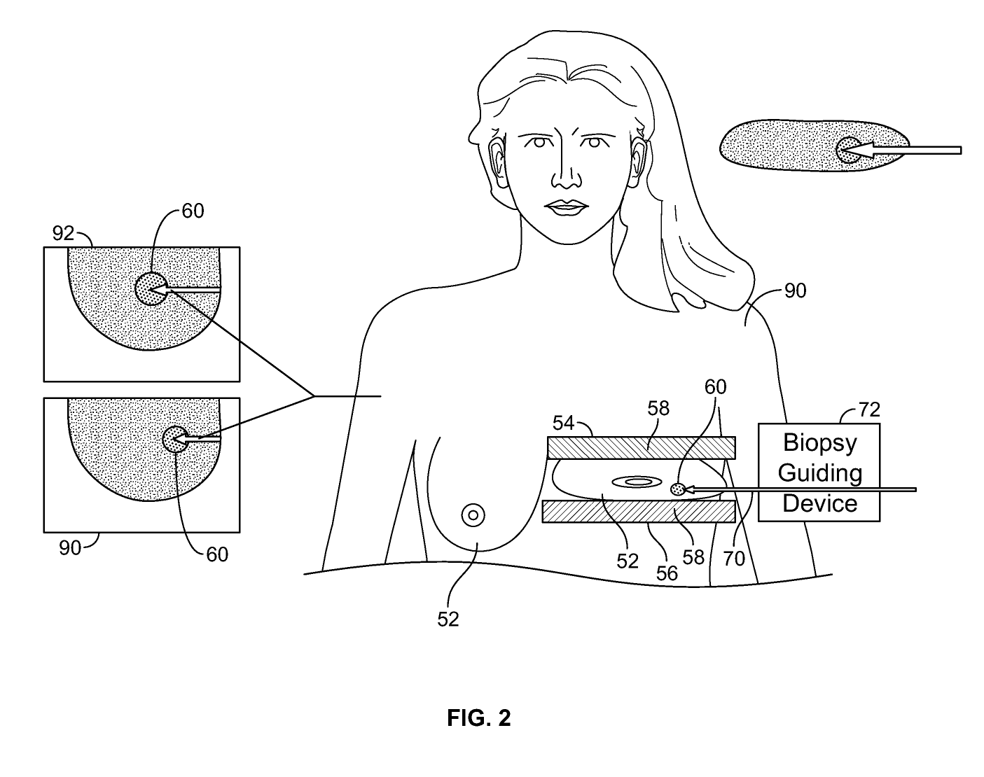

Each of the detectors 54 and 56 has a radiation detection face that is directed towards a structure of interest, for example a lesion 60, within the breast 52. The radiation detection faces are covered by the collimator 58 as described above. An actual field of view (FOV) of each of the detectors 54 and 56 may be directly proportional to the size and shape of the respective imaging detector, or may be changed using the collimator 58. The collimators 58 may be non-slanted collimators as shown in FIG. 1 (with the collimator openings generally perpendicular to the detection face of the detectors 54 and 56) or may be slated collimators having slanted openings as shown in FIG. 2 (with the collimator openings not perpendicular to the detection face of the detectors 54 and 56)/

A motion controller unit 64 may control the movement and positioning of the gantry 62 and/or the detectors 54 and 56 with respect to each other to position the breast 52 within the FOVs of the imaging detectors 54 and 56 prior to acquiring an image of the breast 52. The controller unit 64 may have a detector controller 66 and gantry motor controller 68 that may be automatically commanded by a processing unit 74, manually controlled by an operator, or a combination thereof. The gantry motor controller 68 and the detector controller 66 may move the detectors 54 and 56 individually with respect to the breast 52, with the distance between the detectors 54 and 56 and the orientations thereof registered by the controller 64 and used by the processing unit 74 during data processing. In some embodiments, motion is manually achieved and the controller 64 is replaced with scales or preferably encoders for measuring at least the distance between the detectors 54 and 56, as well as the orientation and/or the compression force exerted by at least one of the detector 54 and/or 56 on the breast 52.

The detectors 54 and 56 and gantry 62 remain stationary after being initially positioned, and imaging data is acquired, as discussed below. The imaging data may be combined and reconstructed into a composite image comprising 2D images and depth information.

A Data Acquisition System (DAS) 76 receives analog and/or digital electrical signal data produced by the detectors 54 and 56 and decodes the data for subsequent processing in the processing unit 74. A data storage device 78 may be provided to store data from the DAS 76 or reconstructed image data. An input device 82 also may be provided to receive user inputs and a display 84 may be provided to display reconstructed images.

The NM imaging system 50 also includes a location module 88 configured to determine the depth of the lesion 60 in the breast 52. Although FIG. 1 shows the location module 88 as a module, it should be appreciated that the location module 88 can also be a program, software, or the like stored on a computer readable medium to be read by the NM imaging system 50.

In operation, the detectors 54 and 56 in some embodiments are capable of being independently or individually rotated to different angles to provide various images of the breast 52, which in various embodiments, results in the detectors 54 and 56 positioned in a parallel or non-parallel arrangement with respect to each other. In various embodiments, the distance between the two detectors 54 and 56 may be changed to accommodate breasts with different sizes and to immobilize the breast for the duration of data acquisition by applying light pressure. The distance between near faces of the two collimators 58 is registered automatically or manually. In one embodiment, one of the detectors moves while the other remains stationary, for example, the upper detector 54 moves toward the lower detector 56 (as viewed in FIG. 1) to immobilize the breast 52 therebetween. Thus, the detectors 54 and 56 are used to apply an immobilizing force to the breast 52. Accordingly, in one embodiment, the breast 52 is positioned between the detectors 54 and 56 and at least one detector is translated to lightly compress and/or maintain the position of the breast 52 between the detectors 54 and 56. It should be noted that the compression of the breast 52 shown in the various figures is exaggerated for illustration. Thus, the distance between the faces of the two collimators 58 in various embodiments is equal to the thickness of the slightly compressed breast, which is registered by the detectors 54 and 56 and may be used by a data analysis program.

The detectors are then used to provide image data of the breast 52 and one or more lesions 60, for example a breast cancer tumor, within the breast 52. As can be seen, the lesion 60 may be located some depth within the breast, and thus at a different distance from each detector, thereby creating different image data in each of the detectors 54 and 56. Thus, the images from the detectors 54 and 56 may be used to determine a position, as well as a depth of the lesion 60 within the breast 52. For example, the depth of the lesion 60 may be calculated based on simple geometry and then used for determining a direction for insertion of a biopsy tool, illustrated as a biopsy needle 70, into the breast 52. It should be noted that the biopsy needle 70 may be any type of needle or biopsy type device. The movement and positioning of the biopsy needle may be controlled or guided by a biopsy guiding tool 72, which may be any suitable device that allows positioning and insertion of the biopsy needle 70 into the breast 52. For example, the biopsy guiding tool may be in the form of a plate having a net of apertures, each aperture providing for insertion of a probe therethrough, for example, as described in U.S. Pat. No. 6,142,991. However other types or kinds of guiding tools may be used. For example a stereotactic tools, robotic tools, etc. may be used. Other medical instruments also may be guided, for example, tools used for brachytherapy, cryogenic therapy, thermal therapy and RF ablation, laser ablation, Photodynamic therapy, etc.

In accordance with various embodiments, the biopsy needle 70 is configured to be opaque (without use of a radioactive source) for imaging with the detectors 54 and 56. For example, as described in more detail a radiation opaque material such as Tungsten or Lead (or a similar type of material) is provided in combination with the biopsy needle 70. Thus, the three-dimensional (3D) location of one or more lesions 60 within the breast 52, as well as the location of the biopsy needle 70 may be determined.

It should be noted that the detectors may be configured or arranged in different configurations. For example, the detectors 54 and 56 may be titled at opposite angles to each other, which may be the same or different. For example, the detectors 54 and 56 may be titled such that the detectors 54 and 56 are closer together at the portion of the breast 52 in the area by the lesion 60 and farther apart at the portion of the breast 52 where there is no lesion 60. Thus, the breast 52 may be compressed more in the area closer to the lesion 60. However, the lesion 60 may also be in the area that is not as compressed. The movement of the detectors 54 and 56, in particular translation of the detectors 54 and 56, which in one embodiment, is translation of the detector 54, is controlled by a detector controller 66. Thus, in one embodiment, the detector controller 66 controls the amount of pressure applied to the breast 52 by the detector 54 to immobilize the breast 52 between the detector 54 and the detector 56, wherein the detector 56 is stationary along the gantry 62.

In operation, data acquired by the detectors 54 and 56 is provided to an image processing module 86 (which may form part of or be installed in the processing unit 74) and/or the location module 88. The location module 88 is used to determine the 3D location of the lesion 60 within the breast 52, which may be used to guide the biopsy needle 70 into the breast 52 toward the lesion 52. The movement of the biopsy needle 70 may be provided by the biopsy guiding device 72, which may be controlled by a biopsy guiding controller (not shown). The biopsy guiding device 72 and the biopsy guiding controller may be provided using any suitable guiding mechanisms or apparatus and may receive lesion location information prior to and/or during insertion (e.g., location feedback information) of the biopsy needle 72 into the breast 52, which is visible in acquired images without using any radioactive material within or applied to the biopsy needle 70.

In particular, various embodiments provide a radiation opaque medical tool, such as the biopsy needle 70. For example, as shown in FIG. 2, a radiation opaque biopsy needle 70 may be provided, wherein the entire needle includes a radiation opaque surface or has parts of the bulk of the needle made from radiation opaque material. In one embodiment, the outer surface of the biopsy needle 80 may be fowled or coated with a radiation opaque material such as Tungsten. In other embodiments, as described herein, only a portion of the biopsy needle 80 is formed or coated with a radiation opaque material, such that a pattern is defined by the placement of the radiation opaque and radiation transparent material.

In operation, breast planar images 90 and 92 of the breast 52 of a patient 90 are acquired that includes the apparent location of the lesion 60, such as on the detectors 54 and 56 (shown in FIG. 1), respectively. The breast planar imaging of the 3D object (in the illustrated embodiment, the breast 52) or a projection of the 3D object used for later image reconstruction is composed of radiation that arrives from a lesion isotope uptake or normal background uptake in healthy tissue. The biopsy needle 80, which is radiation opaque (all or a portion of the biopsy needle 80 may be radiation opaque), blocks some of the emission of gamma rays from the lesion 60 or from the normal background uptake such that the biopsy needle 80 is visible in the images 90 and 92. For example, the location and orientation of the biopsy needle 80 may be determined as described in more detail herein.

In some embodiments, the thickness of the breast 52 during the imaging process as compressed or held between the detectors 54 and 56 is about 5 centimeters (cm). When the biopsy needle 80 is inserted within the breast 52, the radiation opaque properties of the biopsy needed 80, such as formed from, coated with and/or having a rod inserted therein, of a radiation opaque material, blocks or absorbs the radiation, such as from the lesion isotope uptake or normal background, such as blocked from the volume above or below the breast 52. In FIG. 2, the radiation from the lesion 60 is greater than the background radiation from the breast 52. However, as can be seen, the radiation opaque properties of the biopsy needle 80 block or absorb radiation (e.g., gamma rays), such as using radiation opaque material of the biopsy needle 80.

In various embodiments, the contrast between the opaque area and the regular environment is used to localize the biopsy needle 80 as this contrast is proportional to the ratio between the breast thickness and the detector to needle object thickness. Contrast to allow identification of the biopsy needle is achieved in various embodiments when the biopsy needle 80, in particular, the radiation opaque portion of the biopsy needle, which may be the entire biopsy needle 80 or a portion thereof, is located up to half of the breast thickness away from the detector 54 and/or 56. In the embodiment illustrated in FIGS. 1 and 2, using the two detectors 54 and 56 that are positioned on sides of the breast 52 (shown on opposite sides of the breast 52), the position of the biopsy needle 80 at any point within the breast 52 will be closer than half the breast thickness to at least one of the detectors 54 and/or 56 allowing the radiation opaque material of the biopsy needle 80 to be identified and located on at least one of the detectors 54 and/or 56.

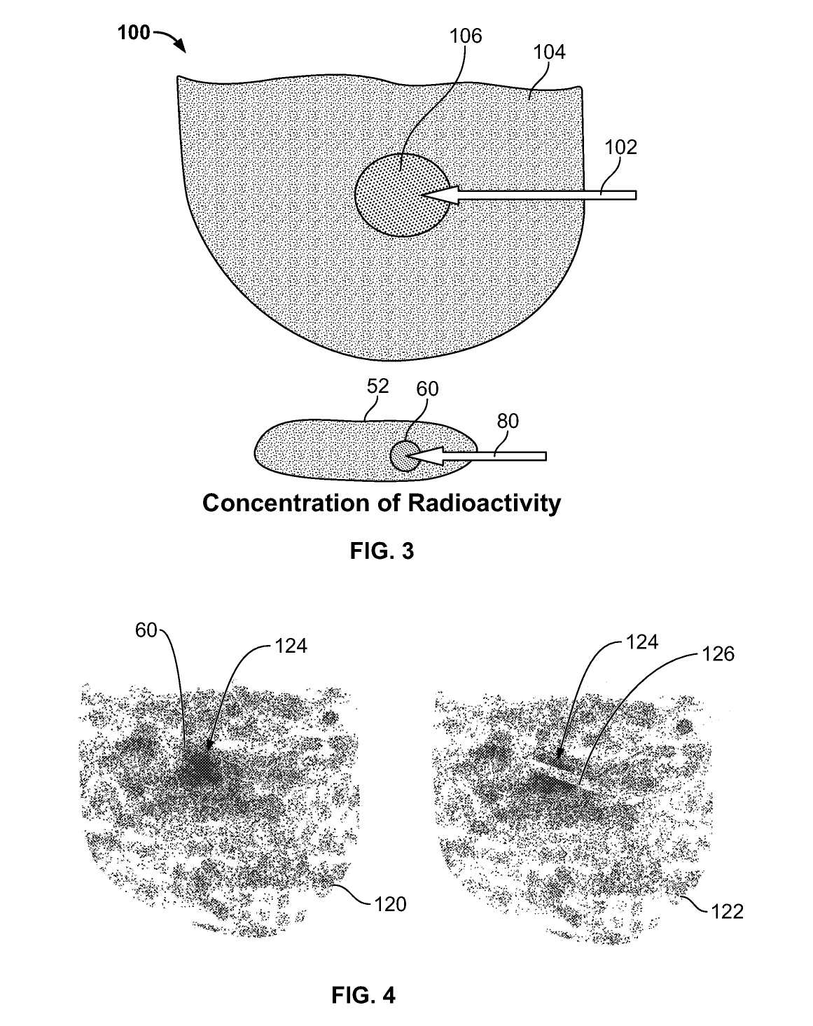

As illustrated in FIG. 3, the concentration of radioactivity is greater from the lesion 60 than the breast 52. In particular, the dots in the image 100 represent the detected gamma photons emitted by radioactivity within the breast 52. As can be seen, the events (e.g., gamma emission counts) are random in the image 100. However, in various embodiments, the concentration of events is different in the area 102 of the biopsy tool 80 than the background concentration 104 and the lesion concentration 106 as a result of the radiation blocking or absorption by the biopsy needle 80 (represented by the smaller concentration of dots within the outline of the biopsy needle 80). In various embodiments, one or more image processing techniques, for example, a pattern recognition technique, correlation, statistical analysis or linear regression approach may be used to identify the lower concentration of radioactivity, thereby localizing the biopsy needle 80. Thus, as shown in FIG. 4, illustrating an image 120 of a breast within the biopsy needle 80 inserted therein and an image 122 of the breast with the biopsy needle 80 inserted, an area 124 of higher concentration of radioactivity corresponds to the lesion 60. As can be seen in the image 122, the radiation opaque properties of the biopsy needle 80 blocks or absorbs the radioactivity such that a reduced number of counts are recorded in the area 126 corresponding to the biopsy needle 80. As can be seen, the biopsy needle 80 is visible in both the lesion 60 and within the breast 52 outside of the lesion 62 (less visible outside the lesion 62 due to the lower concentration of background radioactivity in the breast 52 compared to within the lesion 62).

It should be noted that in planar images, the concentration of radioactivity may be determined from the density of the number of emitted photon counts. In the SPECT (or PET), a more complex determination is used. For example, SPECT breast imaging may be performed as described in U.S. Patent Application Publication 2003/0197127, entitled “SPECT for Breast Cancer Detection”.

It also should be noted that in PET, the penetration is large and scatter data cannot be used as described in more detail herein. It further should be noted that although various embodiments are described in connection with a breast application, various embodiments may be used in non-breast applications, such as for localizing or treating lesions in different regions of the body.

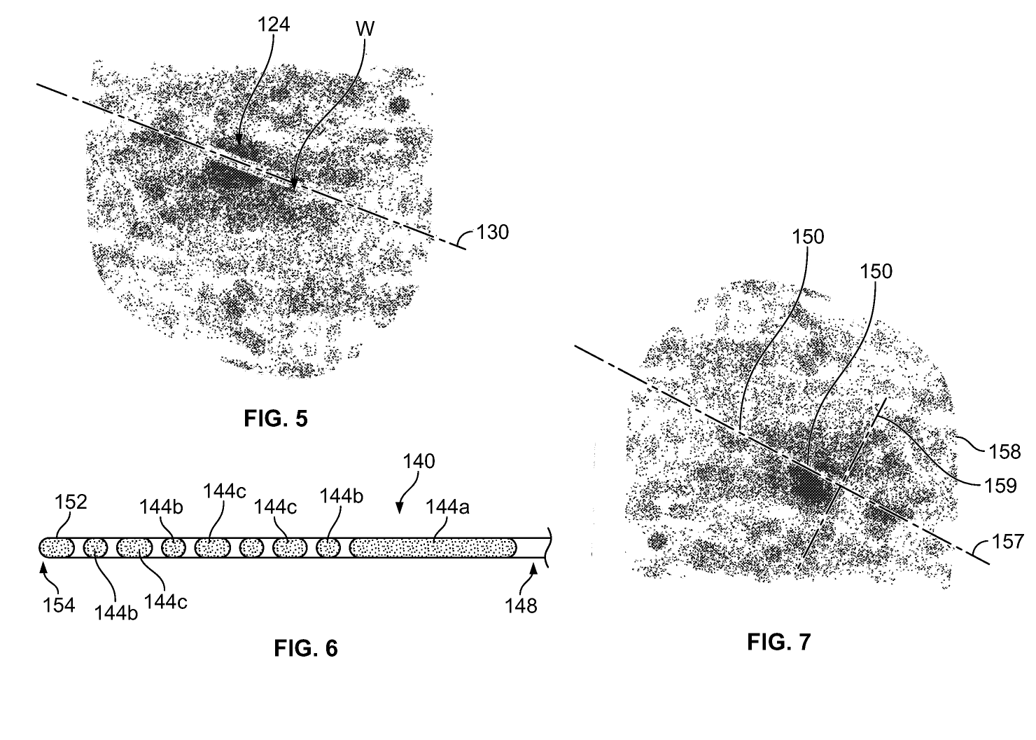

In various embodiments, different types of image processing may be performed. For example, as illustrated in FIG. 5, a linear regression approach or maximum likelihood technique may be used to identify the biopsy needle 80. For example, in various embodiments, the biopsy needle 80 is assumed to be straight in 3D, represented by the line 130. With the thickness of the biopsy needle 80 known, a pattern recognition algorithm may be used to locate and identify a line of known thickness having a lower concentration of radioactivity, for example, a lower concentration of event counts. As can be seen, across the width W of the imaged biopsy needle 80, the concentration of radioactivity goes from a higher concentration from one side of the width to a lower concentration within the width and back to a higher concentration on the other side of the width.

Thus, various embodiments may use image processing techniques to identify the location of the biopsy needle 80 knowing the biopsy needle 80 is straight, such as by linear approximation or a maximum likelihood method. For example, such methods may be used in a view with small signal contrast, large noise and short data acquisition time.

Variations and modifications are contemplated. For example, as shown in FIG. 6, a biopsy needle 140 may be provided that includes an opaque pattern to allow localization of the biopsy needle 140 including a tip 142 of the biopsy needle 140 (instead of forming or coating the entire biopsy needed using a radiation opaque material as in the biopsy needle 80). It should be noted that although the various embodiments have been described in connection with a biopsy needle, the various embodiments may be implemented in connection with any diagnosis tool and/or treatment tool (e.g., cryo-ablation or RF ablation).

As can be seen in FIG. 6, the biopsy needle 140 includes a pattern defined by radiation opaque regions 144. It should be noted that the radiation opaque regions 144 may be formed in different ways. For example, the biopsy needle 140 may be formed from a metal (e.g., stainless steel) and coated in a pattern on an outside circumference around the stainless steel body as shown in FIG. 6. Thus, for example, rings of radiation opaque material, for example Tungsten, may be coated around the biopsy needle 140. In other embodiments, the radiation opaque regions 144 may be formed from powdered Tungsten that makes a composite material from which the biopsy needle 140 is formed. In other embodiments, beads of Tungsten or Gold, for example, may be provided within a stainless steel tube. In still other embodiments, portions of the biopsy needle 140, namely the radiation opaque regions 144 may be formed from Tungsten, with rest of the biopsy needle 140 formed from a radiation transparent material such as stainless steel. In further embodiments, for example, the biopsy needle 140, or portions thereof, may be formed from a heavy metal, such as Lead, which may be encapsulated in various embodiments.

Thus, as can be seen in FIG. 6, the pattern is defined by the radiation opaque regions 144 having radiation transparent regions 146 therebetween. For example, in the illustrated embodiment, the pattern includes a longer radiation opaque region 144a at a proximal end 148 of the biopsy needle 140, followed by a changing pattern of radiation opaque regions 144, illustrated as alternating circular and oval (or smaller and larger) radiation opaque regions 144b and 144c with radiation transparent regions 146 therebetween. However, it should be appreciated that any pattern may be provided, which may be periodic (such as alternating) or random and have different shapes or designs.

Thus, in various embodiments, as shown in FIG. 7, a pattern recognition algorithm may be used to search for a known pattern, in this embodiment, the known alternating pattern of the radiation opaque regions 144, by identifying areas 150 of lower radioactivity corresponding to the biopsy needle 140. Additionally, the tip 152 at a distal end 154 of the biopsy needle also may be identified using the pattern of radiation opaque regions 144, for example, knowing the number of radiation opaque regions 144b and 144c. Thus, the location of the tip 152 and the direction of the biopsy needle 140 may be determined by searching for a known pattern in an image 158 (as shown in FIG. 7), such as a noisy image. For example, a maximum likelihood approach or other image recognition technique may be used to identify the location of the biopsy needle 140 and the tip 152 with the known pattern of radiation opaque regions 144. For example, as can be seen in FIG. 7, the line 157 represents the location of the length of the biopsy needle 140 and the line 159 represents the determined location of the tip 152, which is identified as the end of the last radiation opaque region 144c in the pattern of radiation opaque regions 144b and 144c. It should be noted that the radiation opaque region 144a allows for an easier identification of the biopsy needle 140, which can then be used to locate the radiation opaque regions 144b and 144c along the length of the biopsy needle 140 (assuming a straight tool).

In still other embodiments, scatter radiation (or lower energy radiation) may be used for localization. Thus, in various embodiments, scatter radiation counts are used to locate the medical tool. The scatter radiation photon counts, which are typically high, have a larger absorption coefficient and may be used, for example, to localize a thin coating of a radiation opaque material, such as on the biopsy needle 70 or 80. It also should be noted that the scatter photons are more evenly distributed and do not generally follow the (possibly irregular) radioactive distribution which is influenced by the biological activity of the tissue. The scattered photons are photons that change a direction of travel and lose energy, such as when encountering a tissue atom (instead of passing through the tissue atom or being absorbed by the tissue atom).

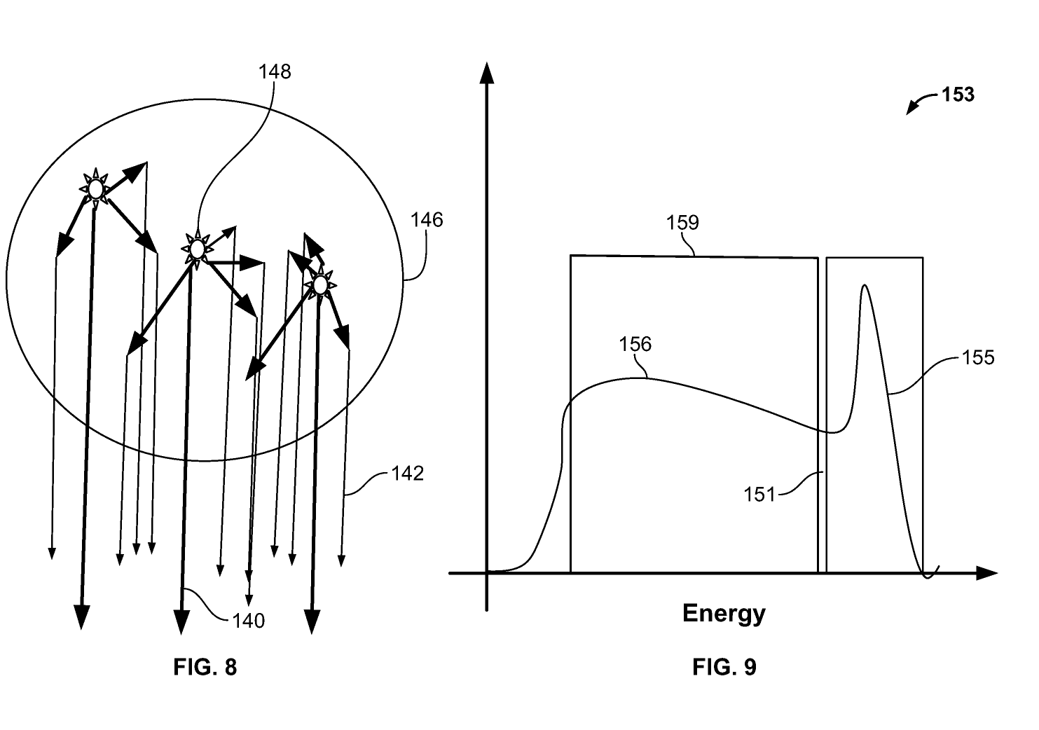

For example, as shown in FIG. 8, scattered photons 142 from radiation sources 148 in the tissue atom 146 are used to localize the medical tool instead of direct photons 140. More particularly, as shown in FIG. 9, nuclear images are generally formed from direct photons within a defined energy window 151 as shown in the graph 153 of FIG. 9 (illustrating an energy profile). Thus, typically the photon counts corresponding to the energy window 151 including the peak 155 are used and the photon counts outside of the energy window discarded or rejected. However, in this embodiment, the scatter photons outside the curve (e.g., along the portion 156 of the curve) are counted within a secondary energy window 159, and used (optionally in combination with peak events counted in energy window 151) to localize the medical tool. The localization of the tool may be performed using pattern recognition techniques or as otherwise described in more detail herein. It should be noted that the counts from the scatter photons are generally less noisy (as there is no pattern from the lesion), which allows various embodiments to localize a thinner medical tool, which would be capable of absorbing the lower radiation scatter photons. Additionally, it should be noted that scatter photons are numerous and thus reduce the statistical noise associated with random photon emission. Also, it should be noted that scatter photons are of lower energy and thus are more susceptible to absorption by the opaque parts of the biopsy tool.

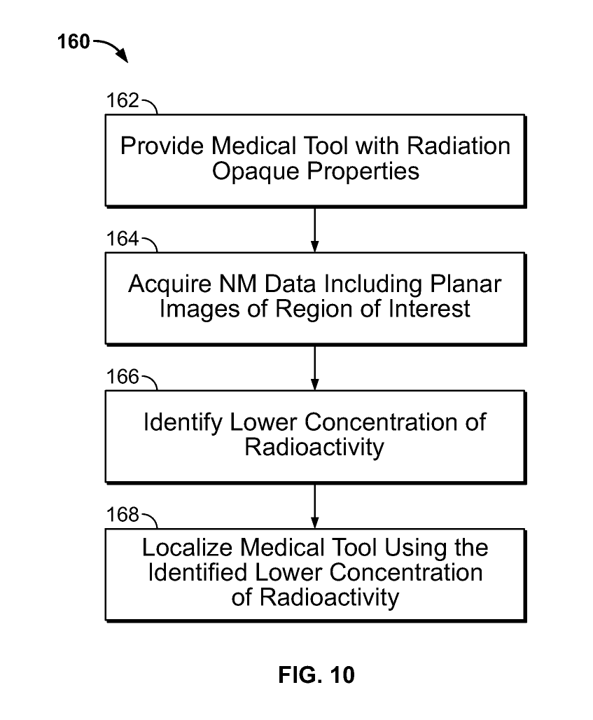

A method 160 for localizing a medical tool (e.g., the biopsy needle 70 or 80) as shown in FIG. 10 may be provided in accordance with various embodiments. The method 160 includes providing a medical tool (e.g., a cylindrical biopsy needle) with radiation opaque properties at 162. For example, all or a portion of the medical tool may be formed from or coated with a radiation opaque material. The radiation opaque material may define a pattern along the medical tool.

At 164, NM data including planar images of a region of interest are acquired. For example, planar (2D) NM breast images may be acquired. It should be noted that in some embodiments, direct photon counts are used. However, in other embodiments, scatter photon counts are used as described herein. From the NM data, a lower concentration of radioactivity is identified at 166, which corresponds to the radiation absorbed by the radiation opaque portion(s) of the medical tool. As described herein, different methods may be used to identify the lower photon count regions or areas, which may include using pattern recognition methods.

Using the lower concentration of radioactivity information, such as the identified lower photon count regions, the medical tool is localized at 168, which may include identifying the length of the medical tool, as well as the tip thereof as described herein.

It should be noted that visualizing the actual location of the tool may be used in various embodiments for reassuring the treating physician that the tool was correctly inserted and to provide feedback for fine adjustment of the positioning of the tool. In some embodiments, the treating physician uses the guiding tool 72 to direct the 70 into a known location in the tissue. The image processing algorithm used in steps 166 and 168 may also use the prior knowledge of the approximate location of the tool to perform a narrow search for the location of the tool. It should be noted that the various embodiments may be used for navigation and provide real-time feedback and treatment (e.g., radioactive beads implantation, cryogenic RF, laser treatment, etc.).

In some embodiments, a radiation opaque sharp insertion tool is used to create a channel within the body. This tool may be made to have high radiation absorbing properties for easy viewing and localization using one or more of the embodiments. The insertion tool is then removed, and a (optionally blunt tip) treatment or biopsy tool is inserted into the created channel. In some embodiments, the insertion tool is sheathed and the sheath remains in place such that the treatment tool may be inserted into the sheath once the insertion tool is removed. Optionally, the sheath is removed after the treatment tool is in place before commencing the treatment of biopsy extraction.

Thus, various embodiments allow for localization of a medical tool without using a tool having radioactive properties (e.g., a radioactivity source therein). Various embodiments localize a medical tool by identifying radiation opaque properties of the tool.

It should be noted that the various embodiments may be used with different imaging systems and methods. For example, the various embodiments may be used with the systems and/or methods described in U.S. Patent Application Publication 2010/0329419 entitled “Gamma Camera for Performing Nuclear Mammography Imaging” and/or U.S. Patent Application Publication 2010/0329418 entitled “System and Method for Performing Nuclear Mammography Imaging”.

It also should be noted that different medical tools may be provided in accordance with one or more embodiments. For example, the medical tool may be a cutting tool with a sheath surrounding the cutting tool, with the sheath remaining within the object when the cutting tool is removed.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid state drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software, which may be a tangible non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A medical tool for use in nuclear medicine imaging, the medical tool comprising:
a body having a length and configured to be inserted within an object; and
one or more radiation opaque regions along at least a portion of the length of the body, the radiation opaque regions blocking gamma ray emission from within the object.

2. The medical tool of claim 1, wherein the one or more radiation opaque regions form part of the body.

3. The medical tool of claim 1, wherein the one or more radiation opaque regions are coated on the body.

4. The medical tool of claim 1, wherein the body is formed from stainless steel and the one or more radiation opaque regions are formed from Tungsten.

5. The medical tool of claim 1, wherein the one or more radiation opaque regions form a pattern along the length of the body.

6. The medical tool of claim 5, wherein the pattern comprises alternating opaque regions having one of different sizes or shapes with radiation transparent regions therebetween.

7. The medical tool of claim 1, wherein the body comprises a biopsy needle.

8. A nuclear medicine (NM) imaging system comprising:
a gantry;
a first nuclear medicine detector mounted to the gantry;
a second nuclear medicine detector mounted to the gantry, wherein the first and second nuclear medicine detectors are configured to acquire planar NM images;
a biopsy guiding tool; and
a biopsy needle configured to be guided within an object between the first and second nuclear medicine detectors, the biopsy needle having one or more radiation opaque properties.

9. The NM imaging system of claim 8, wherein the radiation opaque properties comprises one or more radiation opaque regions along a length of the biopsy needle.

10. The NM imaging system of claim 9, wherein the one or more radiation opaque regions form a pattern along the length of the biopsy needle.

11. The NM imaging system of claim 8, wherein at least a portion of the biopsy needle is formed from a radiation opaque material to define the radiation opaque properties.

12. The NM imaging system of claim 8, wherein at least a portion of the biopsy needle is coated with a radiation opaque material to define the radiation opaque properties.

13. The NM imaging system of claim 8, wherein the biopsy needle is formed from stainless steel and Tungsten defines the one or more radiation opaque properties.

14. A method for localizing a medical tool in nuclear medicine (NM) imaging, the method comprising:
acquiring NM data of a region of interest within an object, the region of interest including a medical tool having one or more radiation opaque regions;
identifying one or more areas of a concentration of radioactivity in an image formed from the NM data, the one or more areas having a lower concentration than at least one of a lesion radioactivity concentration and a background radioactivity concentration, the one or more areas corresponding to the one or more radiation opaque regions; and
localizing the medical tool using the identified one or more areas having the lower concentration of radioactivity.

15. The method of claim 14, further comprising using direct photon count information to identify the one or more areas having the lower concentration of radioactivity.

16. The method of claim 14, further comprising using scatter photon count information to identify the one or more areas having the lower concentration of radioactivity.

17. The method of claim 14, further comprising performing a pattern recognition to identify the one or more areas having the lower concentration of radioactivity corresponding to a pattern of the one or more radiation opaque regions along a length of the medical tool.

18. The method of claim 17, further comprising using the pattern of the one or more radiation opaque regions along a length of the medical tool to identify the medical tool and a tip of the medical tool.

19. The method of claim 14, further comprising using one or a linear regression method or a maximum likelihood method to identify the one or more areas having the lower concentration.

20. The method of claim 14, wherein acquiring the NM data comprises acquiring two-dimensional images of the object using planar gamma cameras on opposite sides of an object.
